(19) United States
(12) Patent Application Publication
(10) Pub. No.: US 2011/0117198 A1
(43) Pub. Date: May 19, 2011

(54) COMPOSITIONS OF SEMI-INTERPENETRATING POLYMER NETWORK

(76) Inventors: Barry James White, East Lothian (GB); Gillian Isabella Rodden, Edinburgh (GB)

(21) Appl. No.: 12/947,082

(22) Filed: Nov. 16, 2010

Related U.S. Application Data
(63) Continuation of application No. 10/583,888, filed on Mar. 23, 2007, now abandoned.

(30) Foreign Application Priority Data
Dec. 23, 2003 (GB) 0329907.0

(51) Int. Cl. A61K 9/00; A61K 31/738; A61P 17/00

(52) U.S. Cl. 424/488; 514/55

(57) ABSTRACT

Novel compositions consisting of semi-interpenetrating network of cross-linked water soluble derivatives of basic polysaccharides and a non-cross-linked component, which is an anionic polysaccharide are provided. Methods for the production of such compositions are also disclosed. Preferably the basic polysaccharide is chitosan or a derivative thereof and the anionic polysaccharide is hyaluronic acid. The compositions can be formed into gels or films, for example, and thus find use in a wide range of medical applications in the fields of dermatology, plastic surgery, urology and orthopaedics.

The present invention relates to hydrogel compositions comprising crosslinked basic polysaccharides formed as semi interpenetrating networks where the basic polysaccharide is crosslinked in the presence of an acidic polysaccharide. In particular, the basic polysaccharide is chitosan or a derivative thereof and the acidic polysaccharide is hyaluronic acid (HA) or a derivative thereof.

Biocompatible polysaccharide compounds are widely used in the biomedical field. To achieve extended residence times in vivo, these compounds are often chemically modified, usually by crosslinking, to form a polymer network.

One of the most widely used biocompatible polymers for medical use is hyaluronic acid (HA). Being a naturally occurring molecule of the same chemical composition in all vertebrates, it is widely accepted to be virtually free from adverse reactions. Hyaluronic acid is an extremely important component of connective tissue and because of its excellent biocompatibility, it has been the subject of many attempts to crosslink the molecule through both its hydroxyl and carboxyl moieties. However, crosslinking does change the chemical structure of the polymer and, for example when used in soft tissue augmentation, cells in the connective tissue which are influenced in their development, migration and proliferation by the milieu in which they are found are exposed to a hyaluronic acid polymer network which is not normally found there.

There is increasing evidence in the scientific literature that exogenously administered natural hyaluronic acid stimulates the synthesis of endogenous hyaluronic acid and, therefore, it can be postulated that a biomaterial comprising a biopolymer network whose residence time in vivo could be modified and which at the same time could deliver exogenous hyaluronic acid in its natural non chemically modified structure over an extended period of time would have potential benefits over crosslinked hyaluronic acid in a number of biomedical applications. It can be further postulated that such a biomaterial could have application as a mimetic of the extra cellular matrix if other polysaccharide components of the natural extra cellular matrix such as chondroitin, dermatan and keratin sulphates were incorporated into the polymer network.

Chitosan, an amino group containing basic polysaccharide, a derivative of the biopolymer chitin, is well reported in the scientific literature as having excellent biocompatibility and is used in a number of biomedical applications.

U.S. Pat. No. 5,977,330 discloses crosslinked N substituted chitosan derivatives where the substitution is by hydroxyacyl compounds that carry carboxylic acids subsequently crosslinked using polyepoxides. No attempt is made to define a semi IPN using these crosslinked derivatives.

U.S. Pat. No. 6,379,702 discloses a blend of chitosan and a hydrophilic poly(N-vinyl lactam). This document does not disclose any crosslinking of the chitosan or the formation of a semi IPN.

U.S. Pat. No. 6,224,893 discloses compositions for forming a semi interpenetrating or interpenetrating polymer networks for drug delivery and tissue engineering whereby the semi IPN is prepared from synthetic and/or natural polymers with a photoinitiator where crosslinking is initiated by free radical generation by electromagnetic radiation.

U.S. Pat. No. 5,644,049 discloses a biomaterial comprising an interpenetrating polymer network whereby one of the components, an acidic polysaccharide, is crosslinked to a second component, a synthetic chemical polymer to create an infinite network. There is no disclosure of crosslinking of acidic polysaccharides with basic polysaccharides.

U.S. Pat. No. 5,620,706 discloses a biomaterial comprising a polyionic complex of xanthan and chitosan for encapsulation and controlled release of biologically active substances. There is no disclosure of covalently crosslinking basic polysaccharides with acidic polysaccharides.

Berger et al. European Journal of Pharmaceutics and Biopharmaceutics, 57 (2004), 19-34 discusses various structures for cross-linked chitosan hydrogels including semi IPN structures.

We have therefore developed a new range of biomaterials, which are based on the formation of a semi IPN with derivatives of cationic polysaccharides which are crosslinked in the presence of anionic polysaccharides under conditions which avoid the formation of ionic complexes between the two polymers and which allow subsequent release of the anionic polysaccharides from the crosslinked network.

Thus, in a first aspect, the present invention provides a composition consisting of a semi interpenetrating polymer network, which comprises at least one crosslinked water soluble derivative of a basic polysaccharide, which has primary and/or secondary amine groups, and a non crosslinked component, which comprises at least one anionic polysaccharide, wherein the anionic polysaccharide resides within the semi interpenetrating polymer network.

A semi interpenetrating polymer network is a combination of at least two polymers formed by covalently crosslinking at least one of the polymers in the presence of but not to the other polymer(s) and having at least one of the polymers in the network as a linear or branched uncrosslinked polymer.

In the context of the present invention, a basic cationic polysaccharide is a polysaccharide containing at least one functional group which is capable of undergoing ionisation to form a cation, eg a protonated amine group, while an acidic anionic polysaccharide is a polysaccharide containing at least one functional group which is capable of undergoing ionisation to form an anion, eg a carboxylate or sulphate ion.

The compositions of the present invention find use as biomaterials, which can be formulated for instance as hydrogels, which in turn can be placed in soft tissue as a mimetic of the extra cellular matrix.

In one embodiment of this aspect of the invention, the water soluble derivative of a basic polysaccharide is a derivative of chitosan, in particular, N-Carboxy methyl chitosan, O-Carboxy methyl chitosan or O-Hydroxy ethyl chitosan or a partially N-acetylated chitosan. The partially N-acetylated chitosan can be produced by partially deacetylating chitin or by reacetylating, chitosan. In any event in one embodiment, the partially N-acetylated chitosan has a degree of acetylation in the range of 45% to 55%.

In another preferred embodiment, the non crosslinked component is hyaluronic acid. In addition, other anionic polysaccharide components of the extra cellular matrix may be included.

The crosslinked component of the composition can be crosslinked using crosslinking agents such as diglycidyl ethers, diisocyanates or aldehydes. In particular, 1,4-Butanedioldiglycidyl ether (BDDE) can be used. The reaction between the epoxide rings at either end of the BDDE molecule and the amine groups on the chitosan chains occurs by nucleophilic attack by the reactive amine groups with subsequent epoxide ring opening as described in “Chitin in Nature and Technology”, R. A. Muzarelli, C. Jeuniaux and G. W. Godday, Plenum Press, New York, 1986, p 303.

The compositions of the present invention can be formed into films, sponges, hydrogels, threads or non woven matrices.

In a second aspect, the present invention provides a method for the preparation of a composition of the invention which comprises crosslinking at least one water soluble derivative of a basic polysaccharide containing primary and/or secondary amine groups, in the presence of at least one anionic polysaccharide, under conditions which avoid protonation of said primary or secondary amine groups on the basic polysaccharide and which also avoid reaction of any other functional group on the water soluble anionic polysaccharide.

As already discussed, the compositions of the present invention can be formed into various forms of biomaterials for use in medical applications. For instance, to produce an injectable hydrogel:

An aqueous solution of a water soluble derivative of a basic polysaccharide containing primary and/or secondary amine groups is formed, to which is added a water soluble anionic polysaccharide. Crosslinking of the basic polysaccharide is then initiated in the presence of a polyfunctional crosslinking agent, under essentially neutral conditions which will only crosslink the primary or substituted amines leaving the anionic polysaccharide entrapped within the crosslinked polymer network.

To Produce a Water Insoluble Film:

An aqueous solution of a water soluble derivative of a basic polysaccharide containing primary and/or secondary amine groups is formed, to which is added a water soluble anionic polysaccharide. A polyfunctional crosslinking agent is then added and the mixture is allowed to evaporate to dryness to allow the crosslinking reaction to take place.

Chitosan becomes soluble in aqueous solutions only when protonated with acids. The polymer thus formed is positively charged and so will interact with negatively charged species such as hyaluronic acid and other polyanions. Such ionic complexes must be avoided in order to form the semi IPN, which is the subject of the present invention.

Thus, chitosan must be solubilised either as an anionic polyelectrolyte or as a non ionic polymer in either a neutral or mildly alkaline medium. As already described, suitable derivatives include N-Carboxy methyl chitosan, O-Carboxy methyl chitosan, O-Hydroxy ethyl chitosan or partially N-acetylated chitosan. In a preferred embodiment, approximately 50% re-acetylated chitosan is used since it can be solubilised in neutral media without protonation of the amine groups. In another preferred embodiment, the re-acetylated chitosan has a degree of deacetylation in the range of 45% to 55% in order to achieve water soluble properties.

The crosslinking reaction in the presence of the polyfunctional crosslinking agent is generally performed under neutral or mildly alkaline conditions, pH range 7 to 8, which ensures that essentially only the primary or secondary amine groups of the basic polysaccharide can react with the crosslinking agent. Thus, crosslinking of the anionic polysaccharide or indeed crosslinking between the acidic and basic polymers is avoided. The degree of crosslinking can be controlled by varying the molar feed ratio of the basic polysaccharide to crosslinking agent. In this way, the release profile of the entrapped anionic polysaccharide can be altered/modified to suit the particular biomedical application in which it is to be used.

Generally, the crosslinking reaction will be carried out around pH 7, preferably between PH 6.8 and 8.

In a third aspect, the present invention provides a biomaterial comprising a composition of the invention.

In a fourth aspect, the present invention provides the use of a composition or of a biomaterial of the invention in medicine.

In a fifth aspect, the present invention provides the use of a composition of the invention in the preparation of a biomaterial. In particular, the biomaterial is for use in dermatology, plastic surgery, urology and in the field of orthopaedics.

Such biomaterials can be formed into films, sponges, hydrogels, threads or non-woven matrices;

Preferred aspects of each aspect of the invention are as for each other aspect mutatis mutandis.

The invention will now be described with reference to the following examples, which illustrate the invention and should not be construed as in any way limiting.

### EXAMPLES

With respect to the following examples a control experiment was carried out using HA and BDDE under the same conditions as for the preparation of all gels only no chitosan was used. There was no evidence of a gel formed after the HA was incubated with BDDE at 50° C. for 3 hours. Therefore we can conclude that under the conditions used to form the semi IPN, the HA does not contribute to gel formation and remains as a linear non crosslinked polymer that is trapped in the crosslinked chitosan matrix.

The water absorption capacity (Q) of the gels and films prepared in the following examples was calculated using the following equation:

$$Q \% = \frac{( \begin{matrix}total wet mass of polymer - \\ total dry mass of polymer\end{matrix} )}{dry mass of crosslinked polymer} \times 100$$

### Example 1

### Gel

Re-acetylated chitosan (2 g, DDA %=54%, Mᵥ=680,000 g/mol) prepared from squid pen chitosan, was hydrated in de-ionised water to give a solution which had a final concentration of 5% weight of polymer. HA (2 g, prepared by fermentation, Hyaltech Ltd) was dissolved in water to give a solution which had a final concentration of 5% weight of polymer. The two solutions were refrigerated overnight to assist the dissolution of the polymers. The two polymer solutions were then mixed together on a high shear mixer and 1,4-butanediol diglycidyl ether (2.5 g, Sigma) was added and stirred into the polymer mixture using a mechanical stirrer. The solution was then crosslinked with mild stirring in a water bath at 50° C. for 3 hours. The gel formed was then immersed in de-ionised water and allowed to swell until it reached constant weight, during which time the water was replaced 4-5 times to remove unreacted residual crosslinker. The water absorption capacity of the gel was 9654% and had a concentration of 10 mg/ml of each polymer. The sample was homogenised on the high shear mixer to enable the gel to be injected from a syringe through a 30 G needle. The mean particle size (D4,3) was 302 μm. The sample had a G′ elastic modulus value of 500 to 600 Pa measured in oscillatory shear over the frequency range from 0.01-10 Hz. An in vitro test was carried out to monitor the release of HA from the gel over a prolonged time period. The same experiment was also carried out in the presence of lysozyme. The results are shown below:

| TIME | % HA RELEASED |
|---|---|
| 0 days | 0.00% |
| 3 days | 1.66% |
| 8 days | 1.57% |
| 11 days | 0.90% |
| 14 days | 0.95% |
| 18 days | 1.25% |
| 21 days | 1.38% |
| 28 days | 1.5% |
| LYSOZYME | |
| 0 days | 0% |
| after 7 days | 1.84% |
| after 13 days | 6.63% |
| after 18 days | 12.9% |
| after 25 days | 16.2% |

### Example 2

### Gel

Re-acetylated chitosan (2 g, DDA %=54%, Mᵥ=680,000 g/mol) prepared from squid pen chitosan, was hydrated in de-ionised water to give a solution which had a final concentration of 5% weight of polymer. HA (1 g, prepared by fermentation, Hyaltech Ltd) was dissolved in water to give a solution which had a concentration of 5% weight of polymer. The two solutions were refrigerated overnight to assist the dissolution of the polymers. The two polymer solutions were then mixed together on a high shear mixer and 1,4-butanediol diglycidyl ether (2.5 g, Sigma) was added and was stirred into the polymer mixture using a mechanical stirrer. The solution was then crosslinked with stirring in a water bath at 50° C. for 3 hours. The gel formed was subsequently immersed in de-ionised water and allowed to swell until it reached constant weight, during which time the water was replaced 4-5 times to remove any unreacted residual crosslinker. The water absorption capacity of the gel was 4551% and gave a concentration of 22 mg/ml for re-acetylated chitosan and 12 mg/ml for HA. The sample was homogenised on the high shear mixer to enable the gel to be injected from a syringe through a 30 G needle. The mean particle size (D4,3) was 255 μm. The sample had a G′ elastic modulus of 2000 to 3000 Pa measured in oscillatory shear over the frequency range from 0.01-10 Hz. An in vitro test was carried out to monitor the release of HA from the gel over a prolonged time period. The same experiment was also carried out in the presence of lysozyme. The results are shown below:

| TIME | % HA RELEASED |
|---|---|
| 0 days | 0% |
| 3 days | 0.014% |
| 8 days | 0.0077% |
| 11 days | 0.088% |
| 14 days | 0.1599% |
| 18 days | 0.337% |
| 21 days | 0.553% |
| 28 days | 0.99% |
| LYSOZYME | |
| 0 days | 0% |
| after 7 days | TLTD |
| after 13 days | 0.22% |
| after 18 days | 0.35% |
| after 25 days | 0.53% |

### Example 3

### Gel

Re-acetylated chitosan (2 g, DDA %=54%, M_{w}≈=750,000 g/mol) prepared from commercial prawn chitosan, was hydrated in de-ionised water to give a solution which had a final concentration of 5% weight of polymer. HA (2 g, prepared by fermentation, Hyaltech Ltd) was dissolved in water to give a solution which had a final concentration of 5% weight of polymer. The two solutions were refrigerated overnight to assist the dissolution of the polymers. The two polymer solutions were then mixed together on a high shear mixer and 1,4-butanediol diglycidyl ether (1.7 g, Fluka) was added and was stirred into the polymer mixture using a mechanical stirrer. The solution was then crosslinked with gentle stirring in a water bath at 50° C. for 3 hours. The gel formed was subsequently immersed in de-ionised water and allowed to swell until it reached constant weight, during which time the water was replaced 4-5 times to remove unreacted residual crosslinker. The water absorption capacity of the gel was 12652% and gave a concentration of 7.9 mg/ml for re-acetylated chitosan and 7.5 mg/ml for HA. When the gel was swollen in phosphate buffered saline (PBS) the final concentration of RAC and HA was 13.54 mg/ml and 12.75 mg/ml respectively. The sample of gel which was swollen in water was homogenised on the high shear mixer to enable the gel to be injected from a syringe through a 30 G needle. The mean particle size (D4,3) was 451 μm. The sample had a G′ elastic modulus value of 1000 Pa measured in oscillatory shear over the frequency range from 0.01-10 Hz. An in vitro test was carried out to monitor the release of HA from the gel over a prolonged time period. The same experiment was also carried out in the presence of lysozyme. The results are shown below:

| TIME | % HA RELEASED |
|---|---|
| 0 days | 0% |
| 5 days | 0.75% |
| 8 days | 0.78% |
| 11 days | 0.78% |
| 15 days | 0.82% |
| 18 days | 0.95% |
| 25 days | 1.36% |
| LYSOZYME | |
| 0 days | 0% |
| after 7 days | 0.91% |
| after 13 days | 1.41% |
| after 18 days | 1.77% |
| after 25 days | 2.4% |

### Example 4

### Gel

O-Hydroxy ethyl chitosan (1 g, Sigma) was hydrated in de-ionised water to give a solution which had a final concentration of 5% weight of polymer. HA (1 g, prepared by fermentation, Hyaltech Ltd) was dissolved in water to give a solution which had a final concentration of 5% weight of polymer. The two solutions were refrigerated overnight to assist the dissolution of the polymers. The two polymer solutions were then mixed together on a high shear mixer and 1,4-butanediol diglycidyl ether (1.5 g, Fluka) was added and was stirred into the polymer mixture using a mechanical stirrer. The solution was then crosslinked with mild stirring in a water bath at 50° C. for 3 hours. The gel formed was subsequently immersed in de-ionised water and allowed to swell until it reached constant weight, during which time the water was replaced 4-5 times to wash away the residual crosslinker. The water absorption capacity of the gel was 8525% and gave a final concentration of 11.7 mg/ml for O-Hydroxy ethyl chitosan and 12.7 mg/ml for HA. The sample was homogenised using a high shear mixer to enable the gel to be injected from a syringe through a 30 G needle. The particle size (D4,3) was 205 μm. The sample had a G′ elastic modulus of 1000 to 2000 Pa measured in oscillatory shear over the frequency range from 0.01-10 Hz.

### Example 5

### Gel

N-Carboxymethyl chitosan (0.6 g, DDA %=85%, Heppe Ltd) was hydrated in de-ionised water to give a solution which had a final concentration of 5% weight of polymer. HA (0.6 g, produced by fermentation, Hyaltech Ltd) was dissolved in water to give a solution which had a final concentration of 5% weight of polymer. The two solutions were refrigerated overnight to assist the dissolution of the polymers. The two polymer solutions were then mixed together on a high shear mixer and 1,4-butanediol diglycidyl ether (0.96 g, Fluka) was added and was stirred into the polymer mixture using a mechanical stirrer. The solution was then crosslinked, with stirring, in a water bath at 50° C. for 8 hours. The gel formed was subsequently immersed in de-ionised water and allowed to swell until it reached constant weight, during which time the water was replaced 4-5 times to remove unreacted residual crosslinker. The water absorption capacity of the gel was 9464% and gave a final concentration of 11 mg/ml for both polymers. The sample was homogenised on the high shear mixer to enable the gel to be injected from a syringe through a 30 G needle. The mean particle size (D4,3) was 218 μm. The sample had a G′ elastic modulus value of 600 to 900 Pa measured in oscillatory shear over the frequency range from 0.01-10 Hz. When the sample was swollen in phosphate buffered saline the concentration of N-Carboxymethyl chitosan and HA was 38 mg/ml and 39 mg/ml respectively.

### Example 6

### Gel

Re-acetylated chitosan (1.9 g, DDA %=54%, Mᵥ=680,000 g/mol) prepared from squid pen chitosan, was hydrated in de-ionised water to give a solution which had a final concentration of 5% weight of polymer. HA (1.9 g, prepared by fermentation, Hyaltech Ltd) was dissolved in water to give a solution which had a final concentration of 5% weight of polymer. The two solutions were refrigerated overnight to assist the dissolution of the polymers. The two polymer solutions were then mixed together on a high shear mixer and 1,4-butanediol diglycidyl ether (0.7 g, Fluka) was added and was stirred into the polymer mixture using a mechanical stirrer. The solution was then crosslinked with stirring in a water bath at 50° C. for 7½ hours. The gel formed was subsequently immersed in de-ionised water and allowed to swell over a period of 2-3 days until it reached constant weight, during which time the water was replaced 4-5 times to remove unreacted residual crosslinker. The water absorption capacity of the gel was 7995% and gave a concentration of 12.5 mg/ml for each polymer. The sample was homogenised on the high shear mixer to enable the gel to be injected from a syringe through a 30 G needle. The mean particle size (D4,3) was 403 μm. The sample had a G′ elastic modulus value of 500 to 800 Pa measured in oscillatory shear over the frequency range from 0.01-10 Hz.

### Example 7

### Film

O-Hydroxy ethyl chitosan (0.2 g) was hydrated in de-ionised water (15 ml). HA (0.1 g) was added to the O-Hydroxy ethyl chitosan solution and stirred until the HA had dissolved. 1,4-Butanediol diglycidyl ether (0.2 g, Sigma) was added and was stirred into the polymer mixture. The solution was then transferred to a Petri dish and was allowed to evaporate for 18 hours during which time a crosslinked film was formed. The film was subsequently immersed in de-ionised water and allowed to swell. The water absorption capacity of the film was 151% and gave a concentration of 660 mg/ml for O-Hydroxy ethyl chitosan and 388 mg/ml for HA. The swelling water was tested for [HA] after 48 hours and resulted in 9.38% of the HA being released. After leaving the film in the swelling water for a further 96 hours no further release of HA was detected.

### Example 8

### Film

Re-acetylated chitosan (0.5 g) was hydrated in de-ionised water at a concentration of 2%. HA (0.5 g, produced by fermentation, Hyaltech Ltd) was dissolved in de-ionised water to give a solution of 2% and the two solutions were placed in the refrigerator to dissolve fully overnight. The two solutions were mixed together and BDDE (0.3 g, Fluka) was added. The polymer mixture was poured into a Petri dish and the water was allowed to slowly evaporate overnight at room temperature forming a crosslinked film. The film was immersed in de-ionised water for two days and was allowed to swell. The WAC of the film was 258% corresponding to a concentration of 383 mg/ml for HA and 387 mg/ml for re-acetylated chitosan. After swelling 0.45% of HA was released from the film. After a further 4 days there was no further detectable release of HA.

1. A composition consisting of a semi interpenetrating network, which comprises at least one crosslinked water soluble derivative of a basic polysaccharide, which has primary and/or secondary amine groups, and a non crosslinked component, which comprises at least one anionic polysaccharide, wherein the anionic polysaccharide resides within the semi interpenetrating polymer network.

2. A composition as claimed in claim 1 wherein the water soluble basic polysaccharide is chitosan or a derivative thereof.

3. A composition as claimed in claim 2 wherein the basic polysaccharide is deacetylated chitin, re-acetylated chitosan, N-Carboxy methyl chitosan, O-Carboxy methyl chitosan or O-Hydroxy ethyl chitosan.

4. A composition as claimed in claim 3 wherein the partially N-acetylated chitosan has a degree of acetylation in the range of 45% to 55%.

5. A composition as claimed in claim 1 wherein the non crosslinked component is hyaluronic acid.

6. A composition as claimed in claim 1 wherein the composition also includes one or other anionic polysaccharide components of the extra cellular matrix.

7. A method for the preparation of a composition as defined in claim 1 which comprises crosslinking at least one water soluble derivative of a basic polysaccharide containing primary and/or secondary amine groups, in the presence of at least one anionic polysaccharide, under conditions which avoid protonation of said primary or secondary amine groups and which also avoid reaction of hydroxyl groups or any other functional group on the anionic polysaccharide.

8. The method as claimed in claim 7 wherein the crosslinking reaction is performed under neutral or slightly alkaline conditions, pH range 7 to 8.

9. A method as claimed in claim 8 wherein the crosslinking reaction is carried out at a ph around 7.

10. A biomaterial comprising a composition as defined in claim 1.

11. The use of a composition as defined in claim 1 in medicine.

12. A method of using the composition as defined in claim 1 for preparing a biomaterial.

13. The method as claimed in claim 12 wherein the biomaterial is for use in dermatology, plastic surgery, urology and in the field of orthopaedics.

14. The method as claimed in claim 13 wherein the biomaterial is formed into a thin film, sponge, hydrogel, thread or non-woven matrix.
